(19) United States
(12) Patent Application Publication
(10) Pub. No.: US 2023/0255711 A1
(43) Pub. Date: Aug. 17, 2023

(54) CALIBRATING AN INSTRUMENT INTERFACE

(71) Applicant: CMR Surgical Limited, Cambridge (GB)

(72) Inventors: Ben Robert Chaplin, Cambridge (US); David William Haydn Webster-Smith, Cambridge (GB)

(73) Assignee: CMR Surgical Limited, Cambridge (GB)

(21) Appl. No.: 18/004,787

(22) PCT Filed: Jul. 13, 2021
(86) PCT No.: PCT/GB2021/051785

§ 371 Date: Jan. 9, 2023

(30) Foreign Application Priority Data
Jul. 14, 2020 (GB) 2010830.4
Feb. 25, 2021 (GB) 2102713.1

(51) Int. Cl. A61B 34/00; A61B 17/00; A61B 34/37

(52) U.S. Cl. CPC A61B 34/77; A61B 17/00; A61B 34/37; A61B 2017/00725

(57) ABSTRACT

A method of calibrating an instrument interface of an instrument in a surgical robotic system, the surgical robotic system comprising a robot having a base and an arm extending from the base to a drive assembly for engaging with the instrument interface to transfer drive to the instrument, the instrument interface being configured to drive joints of the instrument via driving elements, the method comprising: obtaining usage data indicative of usage of a joint of the instrument; comparing the usage data with one or both of a maximum range of joint movement of the joint and a model of expected joint movement of the joint; determining, from the comparison, a calibration offset to adjust a control relationship of a driving element arranged to drive the joint; and adjusting the control relationship of the driving element using the calibration offset so as to calibrate is the instrument interface.

### FIELD OF THE INVENTION

This invention relates to calibrating an instrument interface of an instrument in a surgical robotic system. The calibration uses usage data indicative of usage of a joint of the instrument.

### BACKGROUND

It is known to use robots for assisting and performing surgery. FIG. 1 illustrates a typical surgical robot 100 which consists of a base 108, an arm 102, and an instrument 105. The base supports the robot, and is itself attached rigidly to, for example, the operating theatre floor, the operating theatre ceiling or a trolley. The arm extends between the base and the instrument. The arm is articulated by means of multiple flexible joints 103 along its length, which are used to locate the surgical instrument in a desired location relative to the patient. The surgical instrument is attached to the distal end 104 of the robot arm. The surgical instrument penetrates the body of the patient 101 at a port 107 so as to access the surgical site. At its distal end, the instrument comprises an end effector 106 for engaging in a medical procedure.

FIG. 2 illustrates a typical surgical instrument 200 for performing robotic laparoscopic surgery. The surgical instrument comprises an instrument interface 201 by means of which the surgical instrument connects to the robot arm. A shaft 202 extends between the interface 201 and an articulation 203. The articulation 203 terminates in an end effector 204. In FIG. 2, a pair of serrated jaws are illustrated as the end effector 204. The articulation 203 permits the end effector 204 to move relative to the shaft 202. It is desirable for at least two degrees of freedom to be provided to the motion of the end effector 204 by means of the articulation. The instrument interface is configured to couple to an arm drive assembly of the robot arm to which the instrument is engageable. When the instrument is engaged with the arm, the instrument interface engages with the drive assembly thereby to transfer drive to the instrument.

FIG. 3 illustrates a surgical robot having an arm 300 which extends from a base 301. The arm comprises a number of rigid limbs 302. The limbs are coupled by revolute joints 303. The most proximal limb 302a is coupled to the base by a proximal joint 303a. It and the other limbs are coupled in series by further ones of the joints 303. Suitably, a wrist 304 is made up of four individual revolute joints. The wrist 304 couples one limb (302b) to the most distal limb (302c) of the arm. The most distal limb 302c carries an attachment 305 for a surgical instrument 306. Each joint 303 of the arm has one or more motors 307 which can be operated to cause rotational motion at the respective joint, and one or more position and/or torque sensors 308 which provide information regarding the current configuration and/or load at that joint. Suitably, the motors are arranged proximally of the joints whose motion they drive, so as to improve weight distribution. For clarity, only some of the motors and sensors are shown in FIG. 3. The arm may be generally as described in patent application PCT/GB2014/053523 (WO 2015/132549).

The arm terminates in the attachment 305 for interfacing with the instrument 306. Suitably, the instrument 306 takes the form described with respect to FIG. 2. The instrument has a diameter less than 8 mm. Suitably, the instrument has a 5 mm diameter. The instrument may have a diameter which is less than 5 mm. The instrument diameter may be the diameter of the shaft. The instrument diameter may be the diameter of the profile of the articulation. Suitably, the diameter of the profile of the articulation matches or is narrower than the diameter of the shaft. The attachment 305 comprises a drive assembly for driving articulation of the instrument. Movable interface elements of the drive assembly mechanically engage corresponding movable interface elements of the instrument interface in order to transfer drive from the robot arm to the instrument. One instrument is exchanged for another several times during a typical operation. Thus, the instrument is attachable to and detachable from the robot arm during the operation. Features of the drive assembly interface and the instrument interface aid their alignment when brought into engagement with each other, so as to reduce the accuracy with which they need to be aligned by the user.

The instrument 306 comprises an end effector for performing an operation. The end effector may take any suitable form. For example, the end effector may be smooth jaws, serrated jaws, a gripper, a pair of shears, a needle for suturing, a camera, a laser, a knife, a stapler, a cauteriser, a suctioner. As described with respect to FIG. 2, the instrument comprises an articulation between the instrument shaft and the end effector. The articulation comprises several joints which permit the end effector to move relative to the shaft of the instrument. The joints in the articulation are actuated by driving elements, such as cables. These driving elements are secured at the other end of the instrument shaft to the interface elements of the instrument interface. Thus, the robot arm transfers drive to the end effector as follows: movement of a drive assembly interface element moves an instrument interface element which moves a driving element which moves a joint of the articulation which moves the end effector.

Controllers for the motors, torque sensors and encoders are distributed within the robot arm. The controllers are connected via a communication bus to a control unit 309. The control unit 309 comprises a processor 310 and a memory 311. The memory 311 stores in a non-transient way software that is executable by the processor to control the operation of the motors 307 to cause the arm 300 to operate in the manner described herein. In particular, the software can control the processor 310 to cause the motors (for example via distributed controllers) to drive in dependence on inputs from the sensors 308 and from a surgeon command interface 312. The control unit 309 is coupled to the motors 307 for driving them in accordance with outputs generated by execution of the software. The control unit 309 is coupled to the sensors 308 for receiving sensed input from the sensors, and to the command interface 312 for receiving input from it. The respective couplings may, for example, each be electrical or optical cables, and/or may be provided by a wireless connection. The command interface 312 comprises one or more input devices whereby a user can request motion of the end effector in a desired way. The input devices could, for example, be manually operable mechanical input devices such as control handles or joysticks, or contactless input devices such as optical gesture sensors. The software stored in the memory 311 is configured to respond to those inputs and cause the joints of the arm and instrument to move accordingly, in compliance with a pre-determined control strategy. The control strategy may include safety features which moderate the motion of the arm and instrument in response to command inputs. Thus, in summary, a surgeon at the command interface 312 can control the instrument 306 to move in such a way as to perform a desired surgical procedure. The control unit 309 and/or the command interface 312 may be remote from the arm 300.

The illustrated surgical robot comprises a single robot arm. Other surgical robot systems may comprise a plurality of surgical robots and/or a plurality of robot arms. For example, other example surgical robot systems may comprise a surgical robot with a plurality of robot arms that can each receive and manipulate a surgical instrument, or they may comprise a plurality of surgical robots that each have a robot arm that can receive and manipulate a surgical instrument.

The arm 102 terminates in an attachment, an example of which can be seen in FIG. 4, for interfacing with the instrument. The attachment comprises a drive assembly for driving articulation of the instrument 105. The drive assembly interface 400 interfaces with the instrument interface, an example of which is shown in FIG. 5 at 500. Moveable interface elements 401, 402, 403 of the drive assembly engage corresponding moveable interface elements 502 of the instrument interface in order to transfer drive from the robot arm 102 to the instrument 105. In the example shown in FIGS. 4 and 5, the drive assembly interface elements comprise protruding fins 401, 402, 403 and the interface elements of the instrument interface comprise cups 502 for receiving the fins. The fins and cups can be provided either way round. In some implementations, the drive assembly interface elements comprise cups and the interface elements of the instrument interface comprise fins receivable in the cups. Other ways in which respective drive assembly interface elements and respective instrument interface elements engage with one another may be provided. Linear motion of the drive assembly interface elements causes corresponding is linear motion of the interface elements of the instrument interface.

FIG. 6 is a schematic illustration of a driving mechanism of an instrument interface of a typical instrument. In the driving mechanism illustrated in FIG. 6, the drive assembly interface element 602 comprises a cup and the instrument interface element 604 comprises a fin receivable in the cup 602. The drive assembly interface element 602 (of a robot arm) engages with the instrument interface element 604 of an instrument. In FIG. 6, the driving element 606 is secured at one end to the instrument interface element 604, and at the other end to the end effector 608 via a joint 610. The joint forms part of the articulation 203. The drive assembly interface element 602 engages with the instrument interface element 604 such that motion of the drive assembly interface element is transferred to the instrument interface element leading to corresponding motion of the instrument interface element. The instrument interface element is secured to the driving element such that motion of the instrument interface element 604 is transferred to motion of the driving element 606. Since the driving element is also secured to the end effector 608, motion of the instrument interface element is directly transferred to motion of the end effector. Thus, motion of the drive assembly interface element results in motion of the end effector. The robot arm 102 transfers drive to the end effector 608 of the instrument 105 as follows: movement of a drive assembly interface element 602 moves an instrument interface element 604 which moves a driving element 606 which moves a joint 610 of the articulation 203 which moves the end effector 608. In this example, movement of the drive assembly interface element is transferred to movement of the end effector as a function of a fixed set of parameters (the length of the driving element, the friction of the joint etc.). In this way, the relationship between the position of the drive assembly interface element and the end effector is fixed.

### SUMMARY

This summary is provided to introduce a selection of concepts that are further described below in the detailed description. This summary is not intended to identify key features or essential features of the claimed subject matter, nor is it intended to be used to limit the scope of the claimed subject matter.

According to an aspect of the present invention there is provided a method of calibrating an instrument interface of an instrument in a surgical robotic system, the surgical robotic system comprising a robot having a base and an arm extending from the base to a drive assembly for engaging with the instrument interface to transfer drive to the instrument, the instrument interface being configured to drive joints of the instrument via driving elements, the method comprising: obtaining usage data indicative of usage of a joint of the instrument; comparing the usage data with one or both of a maximum range of joint movement of the joint and a model of expected joint movement of the joint; determining, from the comparison, a calibration offset to adjust a control relationship of a driving element arranged to drive the joint; and adjusting the control relationship of the driving element using the calibration offset so as to calibrate the instrument interface.

Determining the calibration offset may comprise determining, from the comparison, a bias value, indicative of bias of joint usage away from an expected joint usage; and determining the calibration offset from the bias value. The control relationship of the driving element may comprise a positioning of the driving element, and adjusting the control relationship may comprise adjusting the positioning of the driving element using the calibration offset.

The control relationship of the driving element may comprise a configuration of a gearing arranged to drive the driving element, and adjusting the control relationship may comprise adjusting the configuration of the gearing using the calibration offset. Adjusting the control relationship may comprise changing from a first gear ratio to a second gear ratio, moving the driving element, and changing from the second gear ratio back to the first gear ratio.

The control relationship of the driving element may comprise a tension of the driving element, and adjusting the control relationship may comprise adjusting the tension of the driving element using the calibration offset.

The method may comprise obtaining the usage data from a memory and/or from the instrument. The usage data may comprise joint angle data of the joint of the instrument. The usage data may comprise instrument driver position data of an instrument driver configured to drive the joint of the instrument. The usage data may be obtained based on one or more of: a type of instrument, a procedure to be performed using the instrument, a stage in a procedure to be performed using the instrument, an action within a procedure to be performed using the instrument, an ID of a surgical robotic system to which the instrument is attached, and a surgeon ID.

Comparing the usage data with the maximum range of joint movement of the joint may comprise using at least one of a probability density of instrument driver positions and/or joint angular positions, a machine learning algorithm, a statistical analysis technique, and a goodness-of-fit technique.

Determining the calibration offset may comprise identifying a characteristic instrument driver position and/or joint angle from the comparison of the usage data with the respective maximum range of instrument driver position and/or joint movement of the joint, and the method may comprise determining the calibration offset from the characteristic instrument driver position and/or joint angle. Determining the calibration offset may comprise calculating a difference between the characteristic instrument driver position and/or joint angle and a mid-point of the maximum range of instrument driver position and/or joint movement of the joint.

Adjusting a positioning of the driving element may comprise decoupling an instrument interface element from the driving element, modifying the position of the driving element relative to the instrument interface element, and recoupling the interface element to the driving element.

The method may comprise driving the joint using the calibrated instrument interface.

According to another aspect of the present invention there is provided an instrument interface calibrator for calibrating an instrument interface of an instrument in a surgical robotic system, the surgical robotic system comprising a robot having a base and an arm extending from the base to a drive assembly for engaging with the instrument interface to transfer drive to the instrument, the instrument interface being configured to drive joints of the instrument via driving elements, the calibrator being configured to: obtain usage data indicative of usage of a joint of the instrument; compare the usage data with one or more of a maximum range of joint movement of the joint and a model of expected joint movement of the joint; determine, from the comparison, a calibration offset to adjust a control relationship of a driving element arranged to drive the joint; and output the calibration offset for calibrating the instrument interface by adjusting the control relationship of the driving element for the joint.

The calibrator may be configured to determine the calibration offset by determining, from the comparison, a bias value, indicative of bias of joint usage away from an expected joint usage; and to determine the calibration offset from the bias value. The control relationship of the driving element may comprise a positioning of the driving element, and adjusting the control relationship may comprise adjusting the positioning of the driving element using the calibration offset. The control relationship of the driving element may comprise one or more of: a configuration of a gearing arranged to drive the driving element, and adjusting the control relationship comprises adjusting the configuration of the gearing using the calibration offset; a tension of the driving element, and adjusting the control relationship comprises adjusting the tension of the driving element using the calibration offset.

The calibrator may be configured to obtain the usage data from a memory and/or from the instrument. The usage data may comprise instrument driver position data and/or joint angle data of the joint of the instrument. The calibrator may be configured to obtain the usage data based on one or more of: a type of instrument, a procedure to be performed using the instrument, a stage in a procedure to be performed using the instrument, an action within a procedure to be performed using the instrument, an ID of a surgical robotic system to which the instrument is attached, and a surgeon ID.

The calibrator may be configured to compare the usage data with the maximum range of joint movement of the joint by using at least one of a probability density of instrument driver positions and/or joint angular positions, a machine learning algorithm, a statistical analysis technique, and a goodness-of-fit technique.

The calibrator may be configured to determine the calibration offset by identifying a characteristic instrument driver position and/or joint angle from the comparison of the usage data with the maximum range of instrument driver position and/or joint movement of the joint, and the calibrator may be configured to determine the calibration offset from the characteristic instrument driver position and/or joint angle. The calibrator may be configured to determine the calibration offset by calculating a difference between the characteristic instrument driver position and/or joint angle and a mid-point of the maximum range of instrument driver position and/or joint movement of the joint.

Any feature of any aspect described herein may be combined with any other feature of any aspect described herein. Any apparatus feature may be rewritten as a method feature, and vice versa. These are not written out in full merely for the sake of brevity.

### BRIEF DESCRIPTION OF THE DRAWINGS

The present invention will now be described by way of example with reference to the accompanying drawings.

In the drawings:

FIG. 1 illustrates a surgical robot performing a surgical procedure;

FIG. 2 illustrates a surgical instrument;

FIG. 3 illustrates a surgical robot;

FIG. 4 illustrates a drive assembly interface of a surgical robot arm;

FIG. 5 illustrates an instrument interface of a surgical instrument;

FIG. 6 illustrates a driving mechanism of an instrument;

FIG. 7a illustrates drive assembly interface element position data for instrument drivers of an instrument;

FIG. 7b illustrates the drive assembly interface element position data of FIG. 7a after re-zeroing;

FIG. 8 illustrates another example of an instrument interface;

FIG. 9 illustrates an example of a drive assembly interface element interlocking with an instrument interface element;

FIG. 10 illustrates an example of a coupling between a driving element and an instrument interface element;

FIG. 11 illustrates another example of a coupling between a driving element and an instrument interface element;

FIG. 12 illustrates another example of a coupling between a driving element and an instrument interface element;

FIG. 13 illustrates another example of a coupling between a driving element and an instrument interface element;

FIG. 14 illustrates a method of calibrating an instrument interface of an instrument; and

FIG. 15 illustrates components of an exemplary computing-based device.

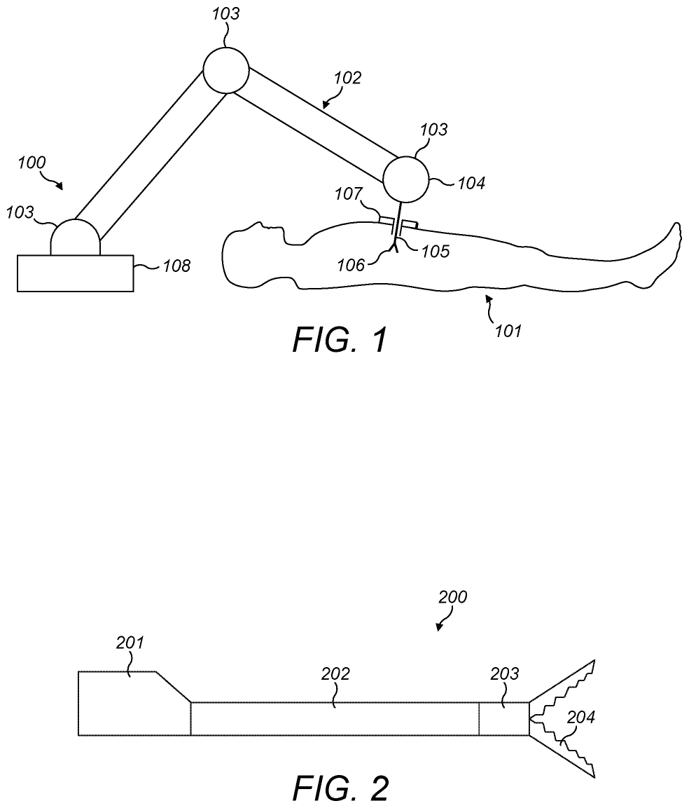

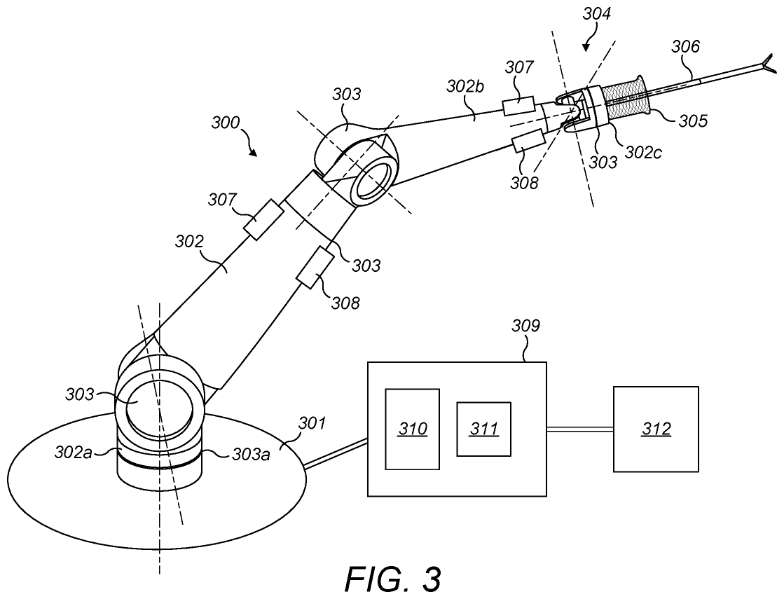

### DETAILED DESCRIPTION

The following description is presented by way of example to enable a person skilled in the art to make and use the invention. The present invention is not limited to the embodiments is described herein and various modifications to the disclosed embodiments will be apparent to those skilled in the art. Embodiments are described by way of example only.

The following description describes the present techniques in the context of surgical robotic systems, though the features described below are not limited to such systems, but may be applied to robotic systems more generally. In some examples, the present techniques may be applied to robotic systems that operate remotely. Robotic systems can include manufacturing systems, such as vehicle manufacturing systems, parts handling systems, laboratory systems, and manipulators such as for hazardous materials or surgical manipulators.

When controlling drive of a joint, such as a rotary joint, using a linear actuator, the range of motion of the joint can be greater than the range of motion of the actuator. An instrument having an end effector can be mounted to a robot arm. The end effector is driven about a plurality of joints of the instrument. The robot arm can generate drive which is transferred to the instrument. Suitably the robot arm comprises a drive assembly, having drive assembly interface elements, which is engageable with an instrument interface, having instrument interface elements, thereby to transfer drive from the robot arm to the instrument. The drive assembly interface elements are linearly movable. The instrument interface elements are correspondingly linearly movable. Movement of the instrument interface elements is arranged to cause movement of portions of the end effector about one or more joints of the end effector.

Thus the linearly actuatable drive assembly interface element drives a linearly moveable instrument interface element to drive rotary motion of an end effector joint. The joint can have a greater range of movement, such as angular movement, than the range of movement of the linearly movable instrument interface element. This means that movement of the instrument interface element across its whole range of movement can be insufficient to control movement of the joint across a portion of its (relatively greater) range of movement.

In such systems, it is useful to be able to identify usage bias of a joint, e.g. where usage of a joint differs from an expected usage of that joint. Such an expected usage of a joint might be generally in the middle of the range of joint movement for that joint. A calibration offset is suitably determined, by which to adjust control of the joint by the instrument interface. This adjustment can permit the instrument interface to more readily be able to control the joint across a desired range of movement of the joint. Thus, usage biases can be identified and the instrument interface calibrated so as to compensate for such usage biases. This improves control of the joint effected by the instrument interface.

In the following, the present techniques will be described with reference to a single instrument interface element coupling to a single driving element for driving a single joint. More generally, the techniques described herein are applicable to multiple instrument interface elements, each coupling to respective driving elements for driving respective joints.

Robotic surgical systems enable collection of telemetry data from surgical procedures. Such telemetry data can be analysed in real-time, e.g. during a surgical procedure, or in non-real-time, such as after a surgical procedure (or after more than one surgical procedure) has been performed. The telemetry data captured by the surgical robotic system suitably comprises data relating to the angular position of one or more joints. This telemetry data permits analysis of system performance and provides evidence for aiding the design of future modifications in the system. The present techniques aim to optimise the range of the instrument's degrees of freedom by analysing collected data relating to the usage of the joints that drive the instrument end effector and recalibrating the instrument on that basis. In one example of a surgical robotic system, the joints of a robotic arm can be numbered progressively from J1 (coupling a first rigid length of the robot arm to a base on which the arm is mounted, i.e. a proximal end of the arm) towards a distal end of the arm. In one implementation, the joints enabling instrument end effector movement are labelled J9, J10 and J11.

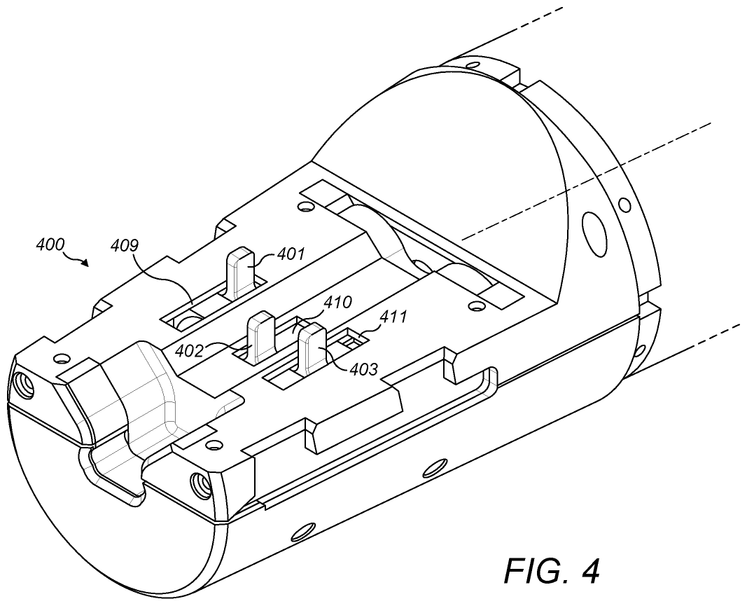

Referring to FIG. 4, drivers for joints J9-J11 can form part of a robot arm drive assembly. The J9-J11 drivers are suitably operable to control movement of the drive assembly interface elements 401, 402, 403. In the example illustrated in FIG. 4, there are three drive assembly interface elements. Each drive assembly interface element is driven by a respective one of the J9-J11 drivers. Each drive assembly interface element is arranged to engage with a respective instrument interface element. There may be fewer or more drive assembly interface elements in other examples, which may be driven by a corresponding number of drivers, and which may be engageable with a corresponding number of is instrument interface elements.

The drive assembly interface elements 401, 402, 403 are drivable along respective linear paths 409, 410, 411 in the drive assembly interface. The instrument drivers (for joints J9-J11) each have a total travel length of 12 mm. That is, each instrument driver is operable to drive its corresponding drive assembly interface element along a linear path of total length 12 mm. Other total travel lengths of the drivers are possible. The total travel lengths of each of the instrument drivers need not be the same. The total travel length of an instrument driver need not be the same as the length of a linear path in the drive assembly interface along which an interface element is driven by that instrument driver. Referring to FIG. 4, it can be seen that the central linear path 410 is shorter than the outer two paths 409, 411. The instrument driver configured to drive the central interface element 402 may be the same as the instrument drivers configured to drive the outer two interface elements 401, 403. Suitably, the total travel length of an instrument driver is the same as or greater than the linear path length along which the interface element driven by that driver moves. In one example, the linear path length along which the central drive assembly interface element is arranged to move is ±2.8 mm from a central zero position. The linear path lengths along which the outer two drive assembly interface elements are arranged to move are each ±6.6 mm from a central zero position.

The (initial) zero position for each instrument driver is found at the midpoint of the travel. For optimal usage of the drivers, the usage distribution should resemble a distribution about the midpoint, such as a symmetrical distribution, e.g. a Gaussian distribution. This means that the drivers would operate mainly in the middle region of travel and less often be required to operate at the outermost regions of travel. By analysing the position data of the instrument drivers which drive the instrument joints J9-J11, biases in the use of at least one of the instrument drivers can be identified.

Once a bias in respect of a joint has been identified, a calibration offset can be determined for use in adjusting the control of that joint by the relevant instrument driver. This approach is useful for those instrument joints which have a greater range of motion than the actuator that drives them. Being able to identify and compensate for usage biases is useful for increasing the range of motion of the joints by the amount determined by the bias (β). The steps followed in order to recalibrate or to “re-zero” the instruments can comprise:
obtaining usage data for a given instrument use situation.
   The usage data can be captured during a procedure. Usage data from earlier procedures can be obtained. Data captured during a procedure can be combined with data obtained in respect of earlier procedures.
   The instrument use situation can be any one or more of use of a particular type of instrument, use of an instrument in a particular procedure or part of a procedure, use of an instrument by a particular surgeon (e.g. by a surgeon associated with a particular surgeon ID in the robotic system), and so on.
determining the usage bias (β). The analysis of the obtained usage data and the determination of the usage bias may be performed in various ways, from simple analysis of the probability density (e.g. mode, median) to machine learning algorithms. The analysis may comprise various techniques within statistical analysis to measure the goodness-of-fit, e.g. of a calculated probability density to a measured probability density.
adjusting a control relationship of a driving element coupling the movable portion of the end effector rotatable about the joint (e.g. a jaw of an instrument) to the instrument interface element.
   For example, with an instrument configured in an alignment configuration (which for a gripper tool may be where the jaws of the instrument are straight and closed), the position of the instrument interface element relative to the driving element (such as a driving cable) can be adjusted. The adjustment can be a mechanical adjustment. The effective driving cable length between the end effector joint and the instrument interface element can be adjusted. The control relationship is suitably adjusted so that the instrument interface element adopts a position that is equal to the initial zero position for the drive assembly interface element coupled to the instrument driver plus the usage bias (β) for that particular joint (related to that drive assembly interface element). This approach enables the drive assembly interface element and the instrument interface element to engage with one another when the instrument is attached to the arm in a manner permitting re-zeroing of the control relationship.
updating the software parameters relating to instrument calibration so that usage bias (β) is accounted for and the kinematics of the instruments are accurate.

The techniques described herein are particularly useful for robotic systems which use linear drives to actuate rotary joints because linear drives have a more limited and well-defined range of motion. Increasing the range of motion of such linear drives would usually compromise the compactness of the drive mechanism. In contrast, rotary drives tend to have a greater range of motion because these could be rotated indefinitely (or at least practically so).

Suitably the control relationship is also at least partially adjustable in software. The software may enable parameters relating to instrument kinematics to be modified. This modification of the parameters can enable the adjustment of the control relationship.

Taking into account the various aspects of the usage data mentioned herein, analysis of the usage data may show evidence of variance of biases from surgeon to surgeon, for each procedure (or specific part of a procedure) or instrument type, for example. It may be desirable to tailor calibration biases to suit the particular needs of each individual case. This can, in some arrangements, be achieved by combining multiple biases. The biases may be weighted according to importance and/or user preference. For example, where a first bias, β₁, is determined in respect of surgeon ID and a second bias, β₂, is determined in respect of a particular procedure, an overall bias, β, can be determined from a weighted combination of β₁ and β₁. In one example, β=aβ₁+bβ₁. In another example, β=aβ₁+(1−a)β₂ (where a≤1). The weighting coefficients, a and b, can be determined empirically or selected in accordance with user preference.

It is possible to calculate or update the usage bias during operation of the instrument. In such cases, the weighting coefficients can vary as a function of one or more of the movement of the instrument, the pose of the end effector of the instrument, and previous movement of that end effector and/or instrument. Conveniently, an analysis of usage data of all of the instrument joints (three in the example discussed herein) permits an assessment of how a user likes to use the system.

The weights may be calculated based on how accurately the usage data maps onto a particular model used to characterise movement of the one or more joints (e.g. using the R² value) and/or the based on the data used to arrive at a given offset, e.g. an amount of data, and/or from how many procedures the data is derived, and/or from how many different robot arms the data is derived.

In some cases, it may be useful to sacrifice range of motion to obtain more torque through is a gear reduction. In these cases, the instrument can be calibrated based on the range of motion expected for which more torque is required, for example a procedure or part of a procedure.

A method of calibrating an instrument interface comprises obtaining usage data indicative of usage of a joint of the instrument. The usage data is compared with a maximum range of joint movement of the joint. The usage data may additionally or alternatively be compared with a model of expected joint movement of the joint, for example in a particular scenario. The model may be a given probability density function. A calibration offset is determined from the comparison to adjust a control relationship of a driving element arranged to drive the joint. The control relationship is adjusted using the calibration offset thereby to calibrate the instrument interface.

Suitably the method can be performed using an instrument interface calibrator. An instrument interface calibrator can be for calibrating an instrument interface of an instrument in a surgical robotic system, the surgical robotic system comprising a robot having a base and an arm extending from the base to a drive assembly for engaging with the instrument interface to transfer drive to the instrument. The instrument interface is configured to drive joints of the instrument via driving elements. The calibrator is configured to: obtain usage data indicative of usage of a joint of the instrument; compare the usage data with a maximum range of joint movement of the joint, and/or compare the usage data with a model of expected joint movement of the joint; determine, from the comparison, a calibration offset to adjust a control relationship of a driving element arranged to drive the joint; and

output the calibration offset for calibrating the instrument interface by adjusting the control relationship of the driving element for the joint. The expected joint movement of the joint is, for example, joint movement expected in a particular scenario. The model may be a given probability density function.

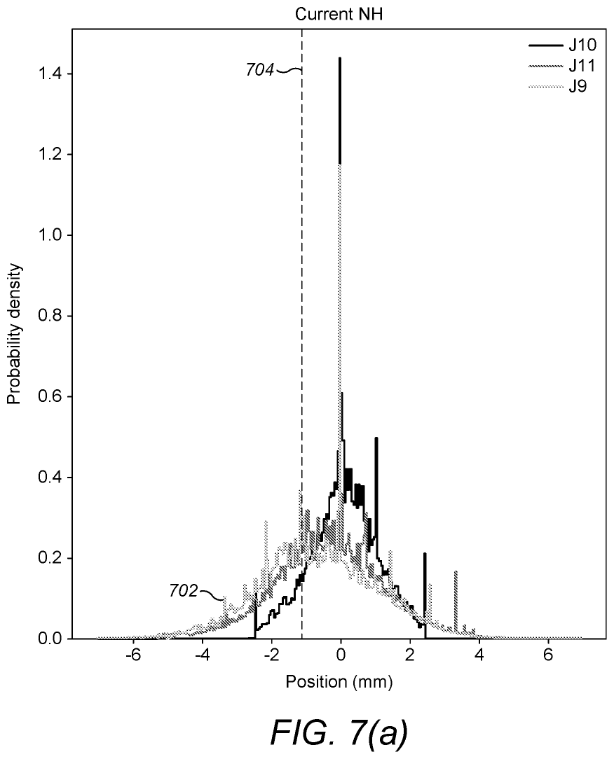

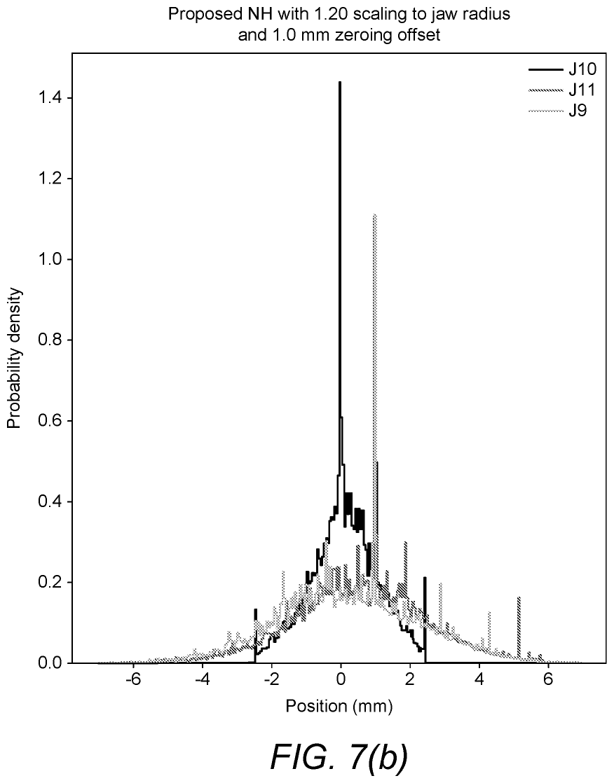

Reference is now made to FIGS. 7a and 7b which show drive assembly interface element position data after 25 hours of surgical use of a needle holder tool (FIG. 7a), and a re-zeroed needle holder tool (FIG. 7b). In FIG. 7a, the probability density data for the J9 driver is illustrated at 702. Fitting a Gaussian distribution to this data leads to identification of a peak at a position (on the x-axis) of approximately −1 mm (shown by dashed line 704). A bias can therefore be identified of 1 mm. Applying an offset of 1 mm to the driver for J9 can cause this probability density data to shift such that the distribution is centred about a zero position of the driver (see FIG. 7b).

Determining the calibration offset can comprise determining, from the comparison, a bias value, indicative of bias of joint usage away from an expected joint usage; and determining the calibration offset from the bias value. In the above example, the bias value is determined as the difference between the peak of a distribution fitted to the usage data and the zero position, at which the usage can be expected to be centred. The calibration offset in this example is the opposite of the bias value, so as to shift the distribution towards the zero position. In other examples, the bias value can be scaled to obtain the calibration offset. The calibration offset may be some other function of the bias value. The calibration offset may be calculated as a function of the usage data of one or more of the instrument joints, and/or of the current position of the end effector.

The bias can additionally or alternatively be calculated using other representative parameters, such as the mean, mode or median of a joint usage value. The calibration offset may be calculated using one or a combination of these mean, mode and median values. The calibration offset may be calculated using a variance value and/or a deviation value, indicating how much variance or deviation, respectively, there is in the data. Such values can comprise, for example, R² or σ values.

The zero position within a range of motion of the instrument driver can be obtained by driving the corresponding drive assembly interface element to one end of the extent of travel. The full extent of the travel is known. This enables the zero position to be set at the position of the drive assembly interface element at the end of the travel plus half the length of travel. In an alternative, the drive assembly interface element can be driven to both ends of the extent of travel and the zero position set at the midpoint. The zero position can be set by having one or more sensors positioned at the midpoint so that whenever the drive assembly interface element is driven past its midpoint it is sensed. The zero position can be set based on the joint, by having one or more sensors positioned at the midpoint of rotation of the joint so that whenever the joint is driven past its midpoint it is sensed.

The control relationship of the driving element arranged to drive the joint can comprise a control relationship effected using the driving element. The control relationship of the driving element arranged to drive the joint can comprise a control relationship associated with the driving of the joint using the driving element. The control relationship can comprise a relationship between an instrument interface and the joint, for example between an instrument interface element of the instrument interface and the joint. The control relationship can comprise a relationship between the instrument interface element and a portion of the driving element and/or between a portion of the driving element and the joint.

Adjusting a control relationship can comprise physically modifying a length of a portion of the driving element linking the instrument interface element to the joint. For example, the physical relationship between the joint and the instrument interface element could be modelled by a linear function where f(x)=p+qx. In this example, q may remain the same and p may change. p can represent an offset in the control relationship. q can represent a gain in the control relationship. Preferably, in this control relationship, p and q are adjustable independently of one another.

The control relationship of the driving element suitably comprises a positioning of the driving element. Adjusting the control relationship comprises adjusting the positioning of the driving element by the calibration offset. The positioning of the driving element is suitably a position relative to the instrument interface, e.g. relative to an instrument interface element.

Adjusting a positioning of the driving element suitably comprises decoupling an instrument interface element from the driving element, modifying the position of the driving element relative to the instrument interface element, and recoupling the interface element to the driving element.

When the instrument interface element is coupled to the driving element, movement of the instrument interface element causes movement of the driving element. The distance of movement of the driving element may be the same as that of the instrument interface element. For example, where the instrument interface element is clamped to a driving cable acting as the driving element, movement of the instrument interface element by 2 mm will directly cause the driving cable to move by 2 mm. Suitably, when the instrument interface element is decoupled from the driving element, movement of the instrument interface element is independent of movement of the driving element. That is, movement of the instrument interface element does not cause movement of the driving element. There is may be a further state in which movement of the instrument interface element causes movement of the driving element by a larger or smaller amount. For example, movement of the instrument interface element by 2 mm in this further state may cause movement of the driving element by more than 2 mm or by less than 2 mm.

Adjustment of the positioning of the driving element can be achieved in various ways. Adjusting the positioning of the driving element can comprise mechanically adjusting a positioning of the driving element. For example, where the interface element is coupled to the driving element by a clamping mechanism such as a screw-tightened clamp, decoupling the instrument interface element from the driving element can be achieved by unscrewing a screw of the screw-tightened clamp, so as to loosen the clamp. When loose, the clamp is able to move relative to the driving element. After repositioning the clamp as desired, the screw can be tightened to securely couple the instrument interface element to the driving element.

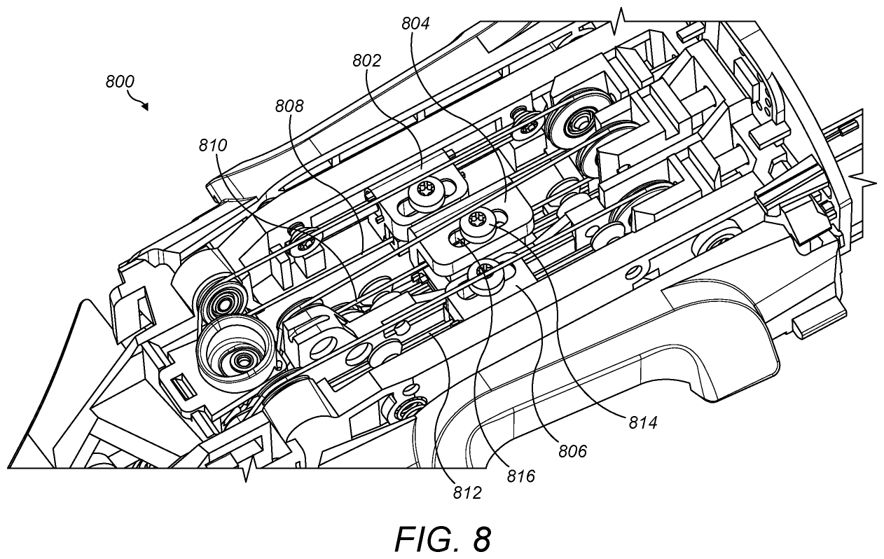

Reference is now made to FIG. 8, which illustrates an example of an instrument interface 800. The instrument interface comprises three instrument interface elements 802, 804, 806, each coupled to a respective driving element 808, 810, 812 by respective coupling mechanisms. In the illustrated example, the coupling mechanisms comprise screws configured to releasably secure the driving elements to the instrument interface elements. As illustrated, the driving elements comprise driving cables. Adjustment of the positioning of the driving element can be achieved by loosening the screw securing the driving cable to the instrument interface element so that the instrument interface element is movable without causing a corresponding movement of the driving cable, moving the instrument interface element relative to the driving cable by the calibration offset, and tightening the screw so that subsequent movement of the instrument interface element will cause corresponding movement of the driving cable.

The screw of the coupling mechanism may engage with a lug attached to the driving cable. Tightening the screw can fix the location of the lug (and the driving cable) relative to the instrument interface element. Loosening the screw can enable relative movement between the lug (and the driving cable) and the instrument interface element. The screw 814 for the central instrument interface element 804 is movable within a channel 816 of the instrument interface element. The length of the channel can determine the amount of the adjustment that can be made in the positioning of the driving cable. The screw can be tightened at any desired position along the channel. This approach permits the calibration offset to smoothly vary. In an alternative, the channel may comprise a plurality of recesses at discrete locations. The screw can be receivable in any one of such recesses. In this alternative, the position of the screw is discrete rather than being continuously variable, and thus the calibration offset can adopt one of a set of discrete values. In such arrangement, the discrete value selected as the calibration offset is suitably the closest discrete value to a calculated calibration offset value. In implementations where the driving element is in the form of a cable having a continuous loop, the cable may be secured relative to the instrument interface elements (802, 804, 806) by means of a releasable coupling such as a tightening screw. The releasable coupling is suitably configured to act on the driving element, e.g. by pressing against the driving element. In this arrangement, the driving cable can be positioned at any desirable position relative to the instrument interface elements.

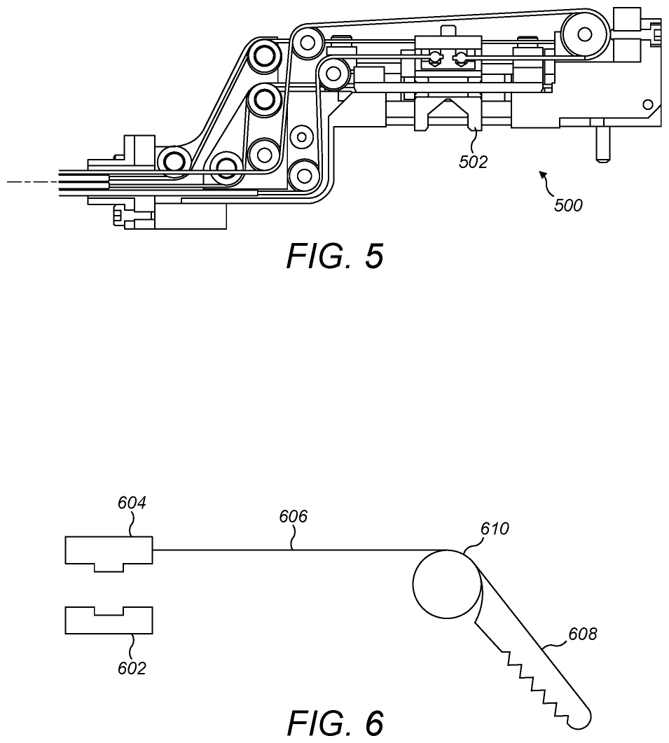

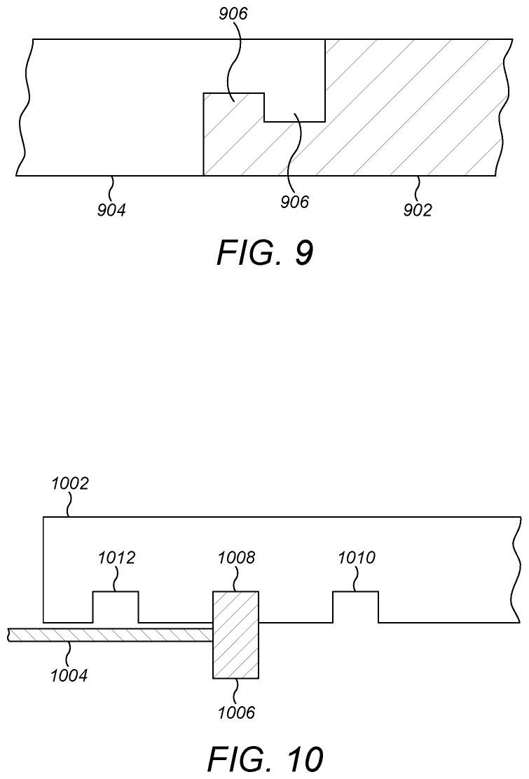

The interfacing between the drive assembly interface element and the instrument interface element may take a form other than that illustrated in FIGS. 4 and 5. For example, the interface elements may be aligned along a longitudinal axis of one or both of a distal portion of the arm and the instrument. In such a configuration, drive can be transferred by a push-pull arrangement, such as a longitudinally-movable rod. The drive assembly interface elements and the instrument interface elements can be of any desired interlocking form, an example of which is illustrated in FIG. 9. FIG. 9 shows a longitudinally extending drive assembly interface element 902 interlocking with a corresponding longitudinally extending instrument interface element 904 by projecting lugs 906 which are receivable into recesses on the elements. In such an arrangement, the coupling between a driving element, such as a driving cable, and the instrument interface element is also suitably achieved by a lug being receivable into a recess of the element. This is illustrated in FIG. 10. The instrument interface element is illustrated at 1002. The driving element comprises a driving cable 1004 and a lug 1006. The lug is received into a recess 1008 of the instrument interface element so as to couple the driving cable to the instrument interface element. The instrument interface element comprises further recesses, two of which 1010, 1012 are shown by way of example. Adjustment of the control relationship can be effected by changing the recess in which the lug 1006 is located. That is, the calibration can be performed by moving the lug from one recess to another. The recesses provide a discrete set of locations at which the lug can be located. Suitably the closest recess to a desired lug position is chosen. The spacing of the lugs can be selected to provide a desired accuracy of calibration. Thus, in is the example illustrated in FIG. 10, the control relationship relates to the lug and recess coupling. Adjusting the control relationship suitably comprises changing the recess in which the lug is located using the calibration offset.

The coupling between an instrument interface element and a driving element may be through a rack and pinion coupling, as illustrated in FIG. 11. An instrument interface element is schematically shown at 1102. A driving cable 1104 is formed of a single loop and passes around two pulleys. One pulley is coupled to a joint 1106 about which a jaw 1108 of a gripper tool is rotatable. The other pulley 1110 is coupled to a pinion 1112. The pinion is engageable with a rack 1114 which is fixed to the instrument interface element 1102. Movement of the instrument interface element 1102 causes corresponding movement of the rack which drives the pinion. Rotation of the pinion causes rotation of the pulley 1110, driving the driving cable to cause rotation of the joint 1106. The instrument interface element can be decoupled from the driving cable by a relative movement of the rack and pinion such that they are separated (i.e. a relative movement in the vertical direction of FIG. 11). Relative movement between the instrument interface element 1102 and the driving cable 1104 can be made in this disengaged configuration. The rack and pinion can be brought back into engagement by a relative movement between them (again, in the vertical direction of FIG. 11) so as to recouple the instrument interface element and the driving cable. In an alternative, the rack can be on the joint side, and the pinion can be on the instrument interface element side. For example, the instrument interface element can couple to a driving element in the form of a loop, which runs over two pulleys, where one pulley is attached to a pinion. Rotation of the pinion can drive a rack which may directly couple to an end effector joint or couple to the end effector joint via a further driving element. Thus, in this arrangement, the control relationship relates to the rack and pinion coupling. Adjusting the control relationship suitably comprises adjusting the configuration of the rack and pinion engagement using the calibration offset. This arrangement can enable the calibration offset to be easily changed when the rack and pinion are disengaged by actuating the rack.

The control relationship of the driving element can additionally or alternatively relate to a configuration of a gearing arranged to drive the driving element. Adjusting the control relationship suitably comprises adjusting the configuration of the gearing using the calibration offset.

is The movement of the driving element may be proportional to that of the instrument interface element. For example, where the instrument interface element is coupled to the driving element by a gearing mechanism, movement of the interface element by 2 mm may cause the driving element to move by more than 2 mm or by less than 2 mm, depending on the gearing. In such examples, adjusting a positioning of the driving element may comprise decoupling an instrument interface element from the driving element, modifying the position of the driving element relative to the instrument interface element, and recoupling the instrument interface element to the driving element, but it need not. In some arrangements, of which a gearing mechanism is an example, it is possible to modify the position of the driving element relative to the instrument interface element by modifying the configuration of the gearing mechanism. For example, adjusting the control relationship can comprise changing from a first gear ratio to a second gear ratio, moving the driving element, and changing from the second gear ratio back to the first gear ratio.

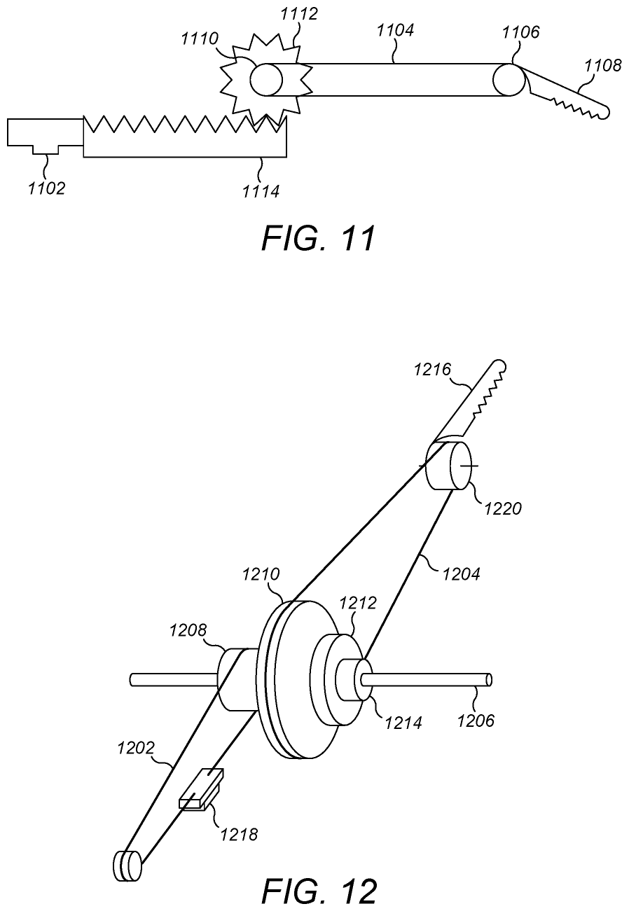

The gearing can comprise pulleys of differing diameters, as illustrated in FIG. 12. The driving element comprises two cable loops, forming a proximal driving element 1202 and a distal driving element 1204. The driving element comprises four pulleys disposed about an axle 1206. The proximal driving element 1202 is constrained about a first pulley 1208 and the distal driving element 1204 is constrained to move about a second pulley 1210. The second pulley 1210 is rotationally fixed relative to the first pulley 1208 such that when the first pulley rotates, the second pulley also rotates. Third and fourth pulleys 1212, 1214 are also positioned on the axle 1206 and are configured to rotate about the axis of the first pulley 1208. The third and fourth pulleys are also rotationally fixed relative to the first pulley such that when the first pulley 1208 rotates, the third and fourth pulleys 1212, 1214 also rotate. Each of the first to the fourth pulleys have a diameter. The diameter of each pulley is different to the diameter of each of the other pulleys. In this example, the fourth pulley 1214 has a diameter that is smaller than the first pulley 1208, but the diameters of the second and third pulleys 1210, 1212 are both larger than the diameter of the first pulley 1208.

A robot arm drive assembly transfers drive to the end effector 1216 of the instrument via an instrument interface element 1218. The distal driving element 1204 can be shifted so that it can be constrained to move about one of the second, third or fourth pulleys. Since each of these pulleys have a different diameter, altering the pulley about which the distal driving element is constrained to move will alter the ratio of the diameter of the driving pulley (the first pulley) to the diameter of the following pulley (one of the second to the fourth pulleys).

The proportion of the displacement of an end of the proximal driving element 1202 which is transferred to displacement of an end of the distal driving element 1204 can be altered by changing the ratio of the diameter of the driving pulley to the diameter of the following pulley. Therefore, changing the pulley about which the distal driving element is constrained to move alters the proportion of motion transferred from the instrument interface element 1218 to the end effector. The illustrated driving element comprises a driving mechanism (not shown) comprising a mechanism configured to allow an operator to change the pulley about which the distal driving element is constrained to move. For example, the driving mechanism may comprise a derailleur, switches or levers.

One or more pulley can be moved relative to another pulley so as to change the tension in the driving elements. Axle 1206, about which the first pulley rotates, can be moved towards or away from the axis of rotation of the pulley 1220 coupled to the end effector 1216. This will accordingly cause the tension in the distal driving element 1204 to decrease or increase, respectively. Decreasing the tension in the driving element can permit the driving element to be more easily moved between the second pulley 1210, the third pulley 1212 and the fourth pulley 1214. Increasing the tension in the driving element can increase the friction between the driving element and the pulley. Increasing the tension in the driving element can permit the driving element to more readily transmit rotation of the respective one of the second, third or fourth pulley to the pulley 1220 coupled to the end effector and thereby to cause movement of the end effector.

Using this arrangement, the control relationship can be adjusted by modifying the displacement of the distal driving element 1204 caused by displacement of the proximal driving element 1202. The displacement of the distal driving element caused by the displacement of the proximal driving element can be selected in dependence on the calibration offset.

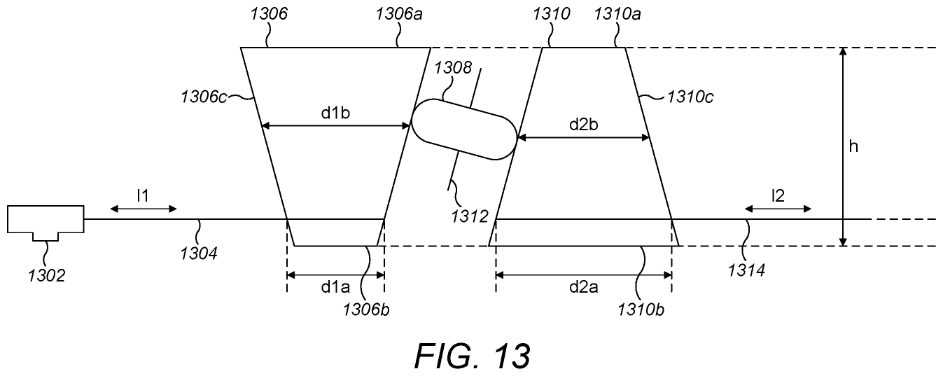

The gearing can comprise two truncated cones, as illustrated in FIG. 13. The instrument interface element 1302 is secured to a proximal driving element 1304. The proximal driving element 1304 engages a first truncated cone 1306. The first truncated cone has two planar circular faces 1306a and 1306b which are parallel to one another, and one curved face 1306c. Circular face 1306a has a diameter greater than that of circular face 1306b. The is proximal driving element 1304 is constrained to move about the first truncated cone 1306. At least a point on the proximal driving element 1304 is secured to the curved face 1306c of the first truncated cone 1306 at or towards the end of the first truncated cone closest to face 1306b. An engagement element 1308 is positioned between the first truncated cone 1306 and a second truncated cone 1310. The second truncated cone 1310 has two parallel planar circular faces 1310a and 1310b and one curved face 1310c. Circular face 1310a has a diameter smaller than that of circular face 1310b. The second truncated cone 1310 is orientated at 180 degrees to the first truncated cone 1306 so as to be in an inverted position relative to the first truncated cone. In other examples, the planar faces of the cones may not be circular, for example they may be generally elliptical. The cone may further be truncated at an angle such that the planar faces of the truncated cone are not parallel. The engagement element 1308 moveably engages face 1306c of the first truncated cone 1306 and face 1310c of the second truncated cone 1310. Faces 1306c and 1310c are parallel to one another at the point at which the engagement element engages them. The engagement element 1308 rotates about an axis 1312. Axis 1312 is parallel to straight lines which entirely intersect faces 1306c and 1310c at the point at which the engagement element engages them. The engagement element has a width equal to the distance between the first and second truncated cones. A distal driving element 1314 engages the second truncated cone 1310. The distal driving element 1314 is constrained to move about the second truncated cone 1310. At least a point on the distal driving element 1314 is secured to the curved face 1310c of the second truncated cone 1310. The proximal and distal driving elements 1304 and 1314 may be secured to the first and second truncated cones 1306 and 1310, respectively using a bead, pin, clip or other adhesive. Alternatively, the curved surfaces 1306c and 1310c of the first and second truncated cones may comprise one or more grooves. The proximal driving element 1304 may sit in a groove of curved surface 1306c of first truncated cone 1306. The distal driving element 1314 may sit in a groove of curved surface 1310c of second truncated cone 1310.

In this example, a robot arm (not shown) transfers drive to the end effector of the instrument as follows: movement of a drive assembly interface element (not shown) moves an instrument interface element 1302 which moves the proximal driving element 1304. Motion of the proximal driving element causes rotation of the first truncated cone 1306. Rotation of the first truncated cone causes rotation of the engagement element 1308 which causes rotation of the second truncated cone 1310. Rotation of the second truncated cone 1310 moves a driving element 1314, which moves a joint causing movement of the end effector.

The ratio of the displacement of the proximal driving element 1304, I1, to the displacement of the distal driving element 1314, I2, depends on the relative rotation of the two truncated cones. The ratio of the displacement of the proximal driving element to the displacement of the distal driving element is therefore a function of: a) the diameter of the first truncated cone 1306 d1a at the point at which the proximal driving element engages the first truncated cone; b) the diameter of the second truncated cone 1310 d2a at the point at which the distal driving element engages the second truncated cone; c) the diameter of the first truncated cone 1306 d1b at the point at which the engagement element 1308 engages the first truncated cone; and d) the diameter of the second truncated cone 1310 d2b at the point at which the engagement element 1308 engages the second truncated cone.

The truncated cones 1306 and 1310 may comprise one or more grooves. The proximal driving element 1304 may sit in a groove of the first truncated cone 1306. The distal driving element 1314 may sit in a groove of the second truncated cone 1310. If each truncated cone comprises a number of grooves, each driving element may be configured to shift between the respective grooves. For example, the positions of the truncated cones may be manually adjusted such that the relative position between each cone and the respective driving element constrained to move around it is altered. The manual mechanism could be a screw that may be tightened. Alternatively, the positions of the cones could be altered using a dedicated driver such as a servomotor. Due to the change in relative position between a driving element and a truncated cone, the driving element may slide on the cone's surface into different grooves. Therefore, there may be a number of points on the cone (values of h) at which the driving element may engage the cone. Therefore, there may be a discrete number of possible values of d1 and of d2a, and a discrete number of possible ratios between them.

The driving elements may be secured to the truncated cones in another way. For example, the driving elements may be secured to the truncated cones using a fixing element such as a bead, clip, pin or using an adhesive. Alternatively, friction between the driving elements and the truncated cones may allow the driving elements to engage the respective truncated cones. The driving elements may be configured to engage the truncated cones at any point on their curved surfaces. The driving element may be configured to engage the curved surfaces at any point along the longitudinal axes of the truncated cones (at any is value of h). For example, the proximal driving element 1304 could be secured to the first truncated cone 1306 at any value of h. Therefore, there may be a continuous range of possible values of d1a. Similarly, there may be a continuous range of possible values of d2a.

The engagement element may comprise one or more protrusions which mesh with one or more grooves or indents in the truncated cones. For example, the first truncated cone may comprise a number of grooves at different points along its longitudinal axis (different values of h). The engagement element may move from one groove to another. For example, the position of the engagement element may be manually adjusted such that the relative position between the engagement element and each truncated cone is altered. The manual mechanism could be a screw that may be tightened. Alternatively, the position of the engagement element could be altered using a dedicated driver such as a servomotor. In some examples, the truncated cones may be required to move to allow the engagement to transition from one groove to another. Therefore there may be a discrete number of points at which the engagement element can engage with the first truncated cone, and a discrete number of possible values for d1b. Similarly there may be a discrete number of points at which the engagement element can engage with the second truncated cone, and a discrete number of possible values for d2b. Therefore there may be a discrete number of possible ratios d1b/d2b.

Alternatively, the engagement element may be configured to engage with the truncated cones in another way such that the engagement element can engage with both truncated cones at any point along their respective longitudinal axes. Therefore, there may be a continuous range of values of d1b and d2b and a large continuous range of possible values of the ratio between these two values.

The engagement element may be configured to move between any two points on the curved surfaces of each of the truncated cones. For example, the engagement element could be a sphere which can rotate to move along the rotation axes of the cones. In an example where the truncated cones do not comprise grooves, the engagement element may engage with the truncated cones due to friction between the engagement element and the cones. The engagement element may be capable of transferring frictional drive in a similar way to a belt.

Using this arrangement, the control relationship can be adjusted by modifying the values of any one of more of d1a, d1b, d2a, d2b. The different values, and ratios of the different values, are suitably selected in dependence on the calibration offset.

Instead of, or as well as, manual calibration using a coupling mechanism (such as a screw clamp coupling mechanism), an automatic adjustment mechanism can be provided. Where manual calibration is to be performed, the instrument will typically be in a non-operational state during the calibration. For example, the instrument can be calibrated before a procedure is started, or before that instrument is mounted to a robot arm during a procedure. The provision of the automatic adjustment mechanism would save time and effort and increase the usefulness of the techniques. In some examples, the instrument can be adjusted by the automatic adjustment mechanism during a procedure. That is, control of the instrument can be updated as a procedure is being performed to optimise use of the instrument performing the procedure. This approach can enable the range of movement of the instrument joints to be maximised whilst potentially reducing the form factor of the instrument interface (and consequently of the arm drive assembly interface and the arm drive assembly).

The automatic adjustment mechanism can comprise further instrument drivers to which the drivers for joints J9-J11 are mounted. Suitably, each of the J9, J10 and J11 drivers are mounted to a respective additional driver. The additional drivers can be driven to move the position of the J9-J11 drivers, and hence the position of the drive assembly interface elements driven by those drivers.

The automatic adjustment mechanism can comprise a disengaging mechanism for disengaging one or more of the instrument interface elements from the respective driving elements. Instrument drivers for the joint for which control is to be recalibrated can be driven to adjust the relative position of the instrument interface element and the driving element. The disengaging mechanism can then cause the instrument interface element and the respective driving element to reengage with one another. The disengaging mechanism can cause a releasing of a clamp of the coupling mechanism to disengage the instrument interface element from the driving element. The disengaging mechanism can cause a tightening of the clamp of the coupling mechanism to reengage the instrument interface element and the driving element. The disengaging mechanism can cause the instrument interface element and the driving element to move between a disengaged configuration in which movement of one does not affect movement of the other and an engaged configuration in which movement of one follows movement of the other.

When the drive assembly interface elements are in the zero position, e.g. at the midpoints of the range of travel, their position will be known, and the instrument interface elements can be correspondingly positioned along their respective ranges of travel so as to permit engagement of the drive assembly interface elements with the instrument interface elements when the instrument is mounted to the robot arm. However, when the instrument has been recalibrated, the new ‘zero’ position of at least one of the instrument interface elements will not be at the original zero position. This means that when the instrument interface elements are all moved back into their original zero position, the configuration of the end effector will differ from the configuration of an end effector of an uncalibrated instrument. For example, the jaws of a gripper tool may no longer be extended straight, but may be at an angle. It is convenient for packing and transportation for the configuration of the instrument to be a known configuration. This can lead to more efficient packing and/or reduce the chances of damage in transit. The instrument suitably comprises a memory for storing calibration information. This calibration information stored locally to the instrument can identify the recalibrated zero positions of the instrument interface elements. The memory can be read by the surgical robotic system before or on attachment of the instrument to the robot arm. The memory can be read wirelessly, via a wireless transceiver on the instrument. The memory can be read through a wired connection established on attaching the instrument to the robot arm. Instead of, or as well as, storing the calibration information at the instrument memory, the calibration information can be associated with an instrument ID of the instrument. On attaching the instrument to the robot arm, the instrument ID can be queried and the calibration information associated with that instrument ID can be retrieved from a memory location accessible to the robotic system. The memory location may be local to or remote from the robotic system. For example, the memory location can be on a remote server. On reading the calibration information, the system can control the end effector joints to be in a desired position, such as in the re-zeroed position.

Where the instrument is attached to the robot arm with the instrument interface elements in the re-zeroed positions, the drive assembly interface elements of the arm drive assembly can be configured to move along their respective paths and try to find the positions at which each drive assembly interface element is engageable with the respective instrument interface element.

Alternatively, the recalibrated instrument can be stored and/or transported with the instrument interface elements in the original zero position, such that the end effector may be in a different configuration (e.g. not straight). This enables the instrument to be easily mounted to the robot arm since the instrument interface elements can thereby easily engage with the drive assembly interface elements. As before, calibration information can be obtained, allowing the system to drive the J9-J11 drivers to positions at which the end effector is ‘re-zeroed’. This can enable convenient mounting of the instrument to the robot arm, whilst permitting the instrument end effector to be inserted through a port with the end effector in a suitable port-insertion position (e.g. for a gripper tool, with the jaws closed and straight).

The control relationship of the driving element can additionally or alternatively comprise a tension of the driving element. Adjusting the control relationship can comprise adjusting the tension of the driving element using the calibration offset. The instrument can comprise an internal biasing mechanism. The internal biasing mechanism is adjustable to modify the effective length of the driving element. For example the driving cable can be split into multiple parts. The cable loop can be closed with a coupling arrangement, such as a screw lock, which can be adjusted to modify the effective cable length.

As described herein, a calibration offset can be determined for an individual joint, e.g. one of J9, J10 and J11 (i.e. a joint controlling movement of an end effector). In some implementations, the calibration offset of one joint may take into account movement and/or a calibration offset of one or more other joints. For example, the calibration offset determined in respect of J9 can be determined based on movement and/or a calibration offset in respect of one or both of J10 and J11. This approach can enable a greater range of motion for one of the joints (e.g. J9) by taking account of one or both of the other joints (e.g. J10 and J11).

The usage data used in the method described herein can, in one implementation, be obtained from a memory. The usage data can be stored in a memory at or associated with the instrument. The memory is suitably a memory on-board the instrument. The usage data may be stored in the instrument memory and communicated to the system via an interface (e.g. together with other instrument data). This arrangement has the advantage that the is usage data for a particular instrument is available irrespective of the robotic system to which that instrument is attached.

The memory can be local to the surgical robotic system. For example, the memory may be provided at a control console of the surgical robotic system. The usage data may be on a memory drive that resides in or is connected to the robotic system (e.g. a console or arm). This approach enables usage data of a plurality of instruments of a single type to be collated at a surgical robotic system to which that plurality of instruments is attached at one time or another. The plurality of instruments of the single type need not all be coupled to the surgical robotic system at the same time. Thus, the usage data for a given instrument (e.g. a gripper tool) can be built up over one or more procedures where one or more gripper tools are used.

The memory can be remote from the surgical robotic system. For example, the memory may be provided at a remote server accessible to the surgical robotic system. The usage data may be on a cloud server and downloaded at the start of the procedure. This approach enables usage data from multiple surgical robotic systems and/or multiple users to be collated. Data from multiple users can be associated with each user's individual usage data. Such usage data can then be accessed by any one surgical robotic system. Hence, a single system can benefit from usage data built up over many procedures.

Combinations of any two or more of these approaches can also be used.

The method can comprise obtaining the usage data from one or more joints of the instrument. E.g., the usage data can be received from the instrument. The usage data can be received from the instrument before operation of the instrument, for example in a set-up procedure. In this case the usage data comprises data relating to one or more previously-performed procedures. The usage data can be received from the instrument during operation of the instrument. The usage data can be or can comprise telemetry data from the instrument, such as from the one or more joints. The usage data thereby obtained is suitably stored in the memory. For example, live usage data received from a joint during a procedure can be stored in an instrument memory, and/or a surgical robotic system memory (e.g. a memory local to the surgical robotic system, such as at a control console), and/or at a remote memory (e.g. a memory at a server remote from the surgical robotic system). The usage data suitably describes movements of the one or more joints of the is instrument.

Suitably, the usage data comprises joint angle data of the joint of the instrument. For example, the usage data can comprise joint angles and a measure of use of the instrument at each joint angle. This can include a measure of time spent at each joint angle, for example at each division of 0.5 degrees, 1 degree, 2 degrees, 5 degrees or the like, within the range of angles of the range of joint movement of that joint. The usage data may comprise instrument driver position data relating to the joint of the instrument. For example, the usage data can comprise instrument driver position and a measure of use of the instrument at each position. This can include a measure of time spent at each position, for example at each division of 0.1 mm, 0.2 mm, 0.5 mm, 1 mm, 1.5 mm, 2 mm or the like, within the range of positions of that instrument driver.

The usage data can be obtained from a kinematics controller or other drive system that controls movement of the one or more joints of the instrument. The usage data can be obtained by measurements of joint angle made using a sensor for sensing the joint angle. The usage data can additionally or alternatively be obtained by analysing control signals sent to a driver for driving the joint. The usage data can additionally or alternatively be obtained using video processing techniques to analyse the pose of the distal end of the instrument, such as the pose of the end effector of the instrument. From this analysed pose, the joint angles associated with the pose can be determined.

The sensor is able to sense joint angle at particular angular increments. The driver is able to drive the joint at particular angular increments. The smallest angular increments at which the joint angle can be sensed and at which the joint angle can be driven may be the same but they need not be. The usage data suitably comprises the measure of use for each joint angle within the whole range of possible joint angles, with each joint angle being separated by the smallest angular increment. This approach can increase the accuracy with which the calibration offset can be determined.

Another approach is to bin data relating to joint usage for angles within sub-ranges of angles. In this way, a histogram can be formed, providing measures of joint usage at each of the sub-ranges. The sub-ranges can be of any desired size, for example, 0.5 degrees of angular rotation of the joint, 1 degree, 2 degrees, 5 degrees and so on. A relatively smaller sub-range size will lead to a more accurate calibration offset, and/or a calibration offset with relatively higher resolution, which can result in a higher accuracy. Use of a smaller sub-range can be expected to lead to relatively more calculations, however. A relatively larger sub-range size may lead to a less accurate calibration offset, and/or a calibration offset with a relatively lower resolution. Use of the larger sub-range can be expected to lead to relatively fewer calculations, which may for example lead to faster processing. A corresponding binning approach can be adopted for the positions of the instrument driver.

The size of the sub-range selected suitably depends on the adjustment of the control relationship of the driving element. Where the control relationship of the driving element is adjustable in discrete stages, a relatively lower resolution calculation of the calibration offset may be sufficient. The further apart the discrete stages, the lower the resolution needs to be. Hence, in these cases, it can be advantageous to make use of potentially faster and/or lower-cost processing by making use of relatively larger sized sub-ranges. An example of a control relationship having discrete stages is the provision of multiple pulleys (as discussed in more detail elsewhere herein) over which a driving cable can pass, each with a different diameter. The diameters of the pulleys represent the discrete nature of the available adjustment. Preferably, the resolution of the calculation of the calibration offset is at least the same as the resolution of the available adjustment. This approach can maximise the resolution available using that adjustment mechanism.

Conversely, where the control relationship of the driving element is smoothly adjustable, a relatively higher resolution calculation of the calibration offset may be more appropriate. Hence, in these cases, it can be advantageous to use relatively smaller sized sub-ranges so as to increase the resolution of the configuration offset that can be calculated. An example of a smoothly adjustable control relationship is the provision of a screw-tightened coupling between an instrument interface element and a driving element (discussed in more detail elsewhere herein). On loosening the screw, the position of the coupling can move by a desired amount that is not restricted to particular positions.

The sub-ranges used to form the histogram may all be the same size (width or angular width). This need not be the case: the size (width or angular width) of one or more sub-range can differ from the size (width or angular width) of one or more other sub-ranges. Where it is known, or expected, that relatively higher joint usage will occur in a given portion of the possible range of movement, the sub-ranges spanning that given portion can is be smaller than sub-ranges outside that given portion. This approach can help reduce processing (compared to the situation where all the sub-ranges are of small size, by using relatively larger sized sub-ranges outside the angular range of most interest) whilst providing for determination of the calibration offset at an increased resolution (compared to the situation where all the sub-ranges are of the relatively larger size).

The usage data is suitably obtained based on one more or more of: a type of instrument, a procedure to be performed using the instrument, a stage in a procedure to be performed using the instrument, an action within a procedure to be performed using the instrument, an ID of a surgical robotic system to which the instrument is attached, and a surgeon ID. This permits usage bias calculations, and thereby the calibration, to be more specific to certain use cases. This tailoring of the calibration can increase accuracy of calibration.

Comparing the usage data with the maximum range of joint movement of the joint suitably comprises using at least one of a probability density of instrument driver positions and/or joint angular positions, a machine learning algorithm, a statistical analysis technique, and a goodness-of-fit technique. Suitably the bias value is determined using at least one of a probability density of instrument driver positions and/or joint angular positions, a machine learning algorithm, a statistical analysis technique, and a goodness-of-fit technique. The bias value can be identified based on a procedure to be performed, a procedure being performed, a procedure already performed, or some combination of more than one of these.

The probability density can comprise a mode, median, or some combination of mode and median. The probability density can comprise any other suitable measure of probability of the respective joint being at a particular instrument driver position and/or joint angle within the maximum range of instrument driver position and/or joint movement of the joint. Thus, the bias value can be identified based on an average of instrument driver positions and/or joint angles for that joint.

The machine learning algorithm can be used to monitor and/or predict instrument driver positions and/or joint angles within the maximum range of instrument driver position and/or joint movement at which the joint is likely to be used. The machine learning algorithm can be trained on instrument joint data relating to one or more of a type of instrument (for is example matching the instrument type of which the instrument interface forms a part), a procedure performed using an instrument (e.g. the same procedure as a procedure to be performed using the instrument), a stage in a procedure performed using an instrument (e.g. the same stage in the procedure as a stage in the procedure to be performed using the instrument), an action within a procedure performed using the instrument (e.g. the same action as an action within the procedure to be performed using the instrument), and a surgeon ID (e.g. the same surgeon ID as that of a surgeon who will perform a procedure using the instrument). In this way, the machine learning algorithm can be used to identify a bias value, or a likely bias value, for a given surgeon carrying out a particular procedure, for example.

Similarly, the statistical analysis technique and/or the goodness-of-fit technique can be based on data relating to a planned procedure, a current procedure, a previous procedure, or some combination of more than one of these. The statistical technique and/or the goodness-of-fit technique can model a predicted or known set of instrument driver positions and/or joint angular positions. For example, a Gaussian model that corresponds to the predicted or known set of instrument driver positions and/or joint angular positions can be determined.

Determining the calibration offset suitably comprises identifying a characteristic driver position and/or joint angle from the comparison of the usage data with the maximum range of instrument driver position and/or joint movement of the joint, and the method comprises determining the calibration offset from the characteristic driver position and/or joint angle. The characteristic driver position and/or joint angle can comprise or be formed based on an instrument driver position and/or a joint angle determined from at least one of the probability density, the machine learning algorithm, the statistical analysis technique and the goodness-of-fit technique.

The characteristic driver position and/or joint angle can comprise or be formed based on a driver position and/or joint angle central to a spread of driver positions and/or joint angles represented by the usage data. The spread of positions and/or angles suitably represents the range of positions and/or angles between the minimum driver position and/or joint angle at which the joint has been used and the maximum driver position and/or joint angle at which the joint has been used. The minimum driver position and/or joint angle may be the lowest driver position and/or joint angle represented by the usage data where joint usage exceeds a minimum usage threshold such as time spent at that driver position and/or joint angle. The maximum driver position and/or joint angle may be the highest driver position and/or joint angle represented by the usage data where joint usage exceeds a further minimum usage threshold such as time spent at that driver position and/or joint angle. The minimum usage threshold and the further minimum usage threshold may be the same but they need not be. The characteristic driver position and/or joint angle can comprise or be formed based on a driver position and/or joint angle mid-way between the minimum driver position and/or joint angle and the maximum driver position and/or joint angle. The characteristic driver position and/or joint angle can comprise or be formed based on a driver position and/or joint angle at which the probability density is at a peak. The characteristic driver position and/or joint angle can comprise or be formed based on the driver position and/or joint angle at which a turning point occurs in the probability density, or at which a turning point occurs in a smoothed probability density. Smoothing the probability density can help increase the accuracy of identifying the characteristic driver position and/or joint angle, for instance by reducing the effect of noise on the identification of the characteristic driver position and/or joint angle. Smoothing the probability density can help increase the accuracy of identifying the characteristic driver position and/or joint angle where driver position and/or joint angle usage data is binned into histogram bins. The characteristic driver position and/or joint angle can comprise or be formed based on the peak of a function, such as a Gaussian function, fitted to the set of predicted or known driver positions and/or joint angles.

The characteristic driver position and/or joint angle may be based on any one or more of the individual driver positions and/or joint angles discussed herein. The characteristic driver position and/or joint angle is suitably calculated as an average or as a weighted average of the individual driver positions and/or joint angles described. The weighting between each of the individual driver positions and/or joint angles is suitably selectable based on one or more of the instrument type, the procedure to be performed, the surgeon ID and so on. Thus, where it is known that a particular procedure involves a complex usage of the instrument, it may be appropriate to weight an individual driver position and/or joint angle determined from the probability density more highly than an individual driver position and/or joint angle determined to be mid-way between the minimum driver position and/or joint angle and the maximum driver position and/or joint angle. This approach can help account for differing usage patterns.

Determining the calibration offset suitably comprises calculating a difference between the characteristic driver position and/or joint angle and a mid-point of the maximum range of driver position and/or joint movement of the joint. E.g., determining the calibration offset comprises calculating a difference between the characteristic driver position and/or joint angle and a position and/or an angle mid-way between the maximum driver position and/or joint angle and the minimum driver position and/or joint angle. This approach permits a determination of whether the spread of positions and/or angles represented by use of the instrument is offset within the range of possible positions and/or angles of use, or central to the range. The determined calibration offset may therefore represent a measure of how much the spread of joint movement is skewed to one side of the range.

In some examples, the calibration offset determined in respect of one joint is based on at least one other joint in the instrument. The calibration offset determined for one instrument joint can be based on all the instrument joints. This approach is useful for pose positions where the usage data for multiple joints shows usage away from an expected joint usage. The calibration of one joint can thereby take account of the calibration and/or usage of another joint.

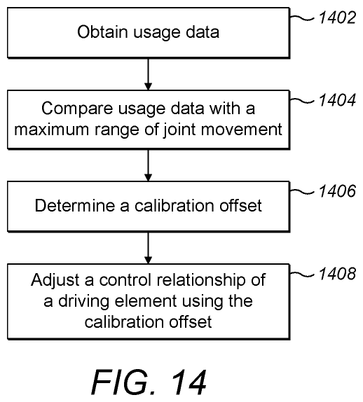

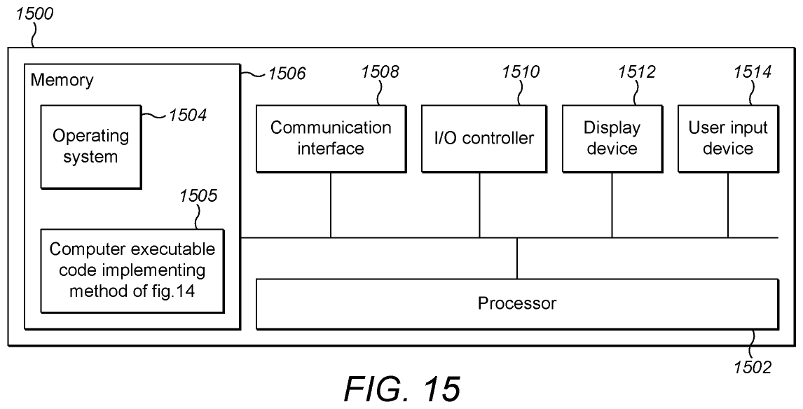

The method of calibrating an instrument interface of an instrument will now be described with reference to FIG. 14. Usage data is obtained (1402). The usage data is indicative of usage of a joint of the instrument. The usage data is compared (1404) with a maximum range of joint movement of the joint. The usage data may additionally or alternatively be compared with a model of expected joint movement of the joint. A calibration offset is determined (1406). The calibration offset is determined from the comparison. The calibration offset is for adjusting a control relationship of a driving element arranged to drive the joint. The control relationship of the driving element is adjusted (1408) using the calibration offset so as to calibrate the instrument interface.

Reference is now made to FIG. 15 which illustrates various components of an exemplary computing-based device 1500 which may be implemented as any form of a computing and/or electronic device, and in which embodiments of the methods and augmentation systems described herein may be implemented. The computing-based device 1500 comprises one or more processors 1502 which may be microprocessors, controllers or any other suitable type of processors for processing computer executable instructions. In some examples, for example where a system on a chip architecture is used, the processors 1502 may include one or more fixed function blocks (also referred to as accelerators) which implement a part of the method in hardware (rather than software or firmware). Platform software comprising an operating system 1504 or any other suitable platform software may be provided at the computing-based device to enable application software, such as software 1505 implementing the method of FIG. 14, to be executed on the device.

The computer executable instructions may be provided using any computer-readable media that is accessible by computing-based device 1500. Computer-readable media may include, for example, computer storage media such as memory 1506 and communications media. Computer storage media (i.e. non-transitory machine-readable media), such as memory 1506, includes volatile and non-volatile, removable and non-removable media implemented in any method or technology for storage of information such as computer readable instructions, data structures, program modules or other data. Computer storage media includes, but is not limited to, RAM, ROM, EPROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing-based device. In contrast, communication media may embody computer readable instructions, data structures, program modules, or other data in a modulated data signal, such as a carrier wave, or other transport mechanism. As defined herein, computer storage media does not include communication media. Although the computer storage media (i.e. non-transitory machine-readable media, e.g. memory 1506) is shown within the computing-based device 1500 it will be appreciated that the storage may be distributed or located remotely and accessed via a network or other communication link (e.g. using communication interface 1508).

The computing-based device 1500 also comprises an input/output controller 1510 arranged to output display information to a display device 1512 which may be separate from or integral to the computing-based device 1500. The display information may provide a graphical user interface. The input/output controller 1510 is also arranged to receive and process input from one or more devices, such as a user input device 1514 (e.g. a mouse or a keyboard). This user input may be used to initiate verification. In an embodiment the display device 1512 may also act as the user input device 1514 if it is a touch sensitive display device. The input/output controller 1510 may also output data to devices other than the display device, e.g. a locally connected printing device (not shown). In the description above actions taken by the system have been split into functional blocks or modules for ease of explanation. In practice, two or more of these blocks could be architecturally combined. The functions could also be split into different functional blocks.

The present techniques have been described in the context of surgical robotic systems, though at least some features described are not limited to such systems, but may be applied to robotic systems more generally. In some examples, the present techniques may be applied to robotic systems that operate remotely. Some examples of situations in which the present techniques may be useful include those that make use of ‘snake-like’ robots for exploration, investigation or repair. In the case of a surgical robot the end effector could be a surgical tool such as a scalpel, surgical cutter, surgical pincer or cauteriser. Robotic systems can include manufacturing systems, such as vehicle manufacturing systems, parts handling systems, laboratory systems, and manipulators such as for hazardous materials or surgical manipulators.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention as defined by the claims.

1. A method of calibrating an instrument interface of an instrument in a surgical robotic system, the surgical robotic system comprising a robot having a base and an arm extending from the base to a drive assembly for engaging with the instrument interface to transfer drive to the instrument, the instrument interface being configured to drive joints of the instrument via driving elements, the method comprising:
obtaining usage data indicative of a usage distribution of a joint of the instrument;
comparing the usage data with one or both of a maximum range of joint movement of the joint and a model of expected joint movement of the joint;
determining, from the comparison, a calibration offset to adjust a control relationship of a driving element arranged to drive the joint; and
adjusting the control relationship of the driving element using the calibration offset so as to calibrate the instrument interface.

2. A method according to claim 1, in which determining the calibration offset comprises determining, from the comparison, a bias value, indicative of bias of joint usage away from an expected joint usage; and determining the calibration offset from the bias value.

3. A method according to claim 1, in which the control relationship of the driving element comprises a positioning of the driving element, and adjusting the control relationship comprises adjusting the positioning of the driving element using the calibration offset.

4. A method according to claim 1, in which the control relationship of the driving element comprises a configuration of a gearing arranged to drive the driving element, and adjusting the control relationship comprises adjusting the configuration of the gearing using the calibration offset.

5. A method according to claim 4, in which adjusting the control relationship comprises changing from a first gear ratio to a second gear ratio, moving the driving element, and changing from the second gear ratio back to the first gear ratio.

6. A method according to claim 1, in which the control relationship of the driving element comprises a tension of the driving element, and adjusting the control relationship comprises adjusting the tension of the driving element using the calibration offset.

7. (canceled)

8. A method according to claim 1, in which the usage data comprises joint angle data of the joint of the instrument.

9. A method according to claim 1, in which the usage data comprises instrument driver position data of an instrument driver configured to drive the joint of the instrument.

10. (canceled)

11. A method according to claim 1, in which comparing the usage data with the maximum range of joint movement of the joint comprises using at least one of a probability density of instrument driver positions and/or joint angular positions, a machine learning algorithm, a statistical analysis technique, and a goodness-of-fit technique.

12. A method according to claim 1, in which determining the calibration offset comprises identifying a characteristic instrument driver position and/or joint angle from the comparison of the usage data with the respective maximum range of instrument driver position and/or joint movement of the joint, and the method comprises determining the calibration offset from the characteristic instrument driver position and/or joint angle.

13. A method according to claim 12, in which determining the calibration offset comprises calculating a difference between the characteristic instrument driver position and/or joint angle and a mid-point of the maximum range of instrument driver position and/or joint movement of the joint.

14. A method according to claim 1, in which adjusting a positioning of the driving element comprises decoupling an instrument interface element from the driving element, modifying the position of the driving element relative to the instrument interface element, and recoupling the interface element to the driving element.

15. (canceled)

16. An instrument interface calibrator for calibrating an instrument interface of an instrument in a surgical robotic system, the surgical robotic system comprising a robot having a base and an arm extending from the base to a drive assembly for engaging with the instrument interface to transfer drive to the instrument, the instrument interface being configured to drive joints of the instrument via driving elements, the calibrator being configured to:
obtain usage data indicative of a usage distribution of a joint of the instrument;
compare the usage data with one or more of a maximum range of joint movement of the joint and a model of expected joint movement of the joint;
determine, from the comparison, a calibration offset to adjust a control relationship of a driving element arranged to drive the joint; and
output the calibration offset for calibrating the instrument interface by adjusting the control relationship of the driving element for the joint.

17. An instrument interface calibrator according to claim 16, in which the calibrator is configured to determine the calibration offset by determining, from the comparison, a bias value, indicative of bias of joint usage away from an expected joint usage; and to determine the calibration offset from the bias value.

18. An instrument interface calibrator according to claim 16, in which the control relationship of the driving element comprises a positioning of the driving element, and adjusting the control relationship comprises adjusting the positioning of the driving element using the calibration offset.

19. An instrument interface calibrator according to claim 16, in which the control relationship of the driving element comprises one or more of:
a configuration of a gearing arranged to drive the driving element, and adjusting the control relationship comprises adjusting the configuration of the gearing using the calibration offset;
a tension of the driving element, and adjusting the control relationship comprises adjusting the tension of the driving element using the calibration offset.

20. (canceled)

21. An instrument interface calibrator according to claim 16, in which the usage data comprises instrument driver position data and/or joint angle data of the joint of the instrument.

22. (canceled)

23. An instrument interface calibrator according to claim 16, in which the calibrator is configured to compare the usage data with the maximum range of joint movement of the joint by using at least one of a probability density of instrument driver positions and/or joint angular positions, a machine learning algorithm, a statistical analysis technique, and a goodness-of-fit technique.

24. An instrument interface calibrator according to claim 16, in which the calibrator is configured to determine the calibration offset by identifying a characteristic instrument driver position and/or joint angle from the comparison of the usage data with the maximum range of instrument driver position and/or joint movement of the joint, and the calibrator is configured to determine the calibration offset from the characteristic instrument driver position and/or joint angle.

25. An instrument interface calibrator according to claim 24, in which the calibrator is configured to determine the calibration offset by calculating a difference between the characteristic instrument driver position and/or joint angle and a mid-point of the maximum range of instrument driver position and/or joint movement of the joint.
